(19) United States
(12) Patent Application Publication
(10) Pub. No.: US 2023/0365808 A1
(43) Pub. Date: Nov. 16, 2023

(54) MODIFIED SILICONE RUBBERS AND METHODS FOR PREPARING THEREOF, ACOUSTIC PERMEABLE COMPONENTS, ULTRASONIC PROBES, AND ULTRASONIC DIAGNOSTIC DEVICES

(71) Applicant: WUHAN UNITED IMAGING HEALTHCARE CO., LTD., Wuhan (CN)

(72) Inventor: Yu LIU, Wuhan (CN)

(73) Assignee: WUHAN UNITED IMAGING HEALTHCARE CO., LTD., Wuhan (CN)

(21) Appl. No.: 18/318,756

(22) Filed: May 17, 2023

Related U.S. Application Data
(63) Continuation of application No. PCT/CN2022/143488, filed on Dec. 29, 2022.

(30) Foreign Application Priority Data
May 13, 2022 (CN) 202210521312.1

(51) Int. Cl. C08L 83/04

(52) U.S. Cl. CPC C08L 83/04

(57) ABSTRACT

Disclosed is modified silicone rubber and a preparation method thereof, an acoustic permeable component, an ultrasonic probe, and an ultrasonic diagnostic device. The modified silicone rubber may comprise: a cured matrix formed by curing a room temperature vulcanized (RTV) silicone rubber; and polystyrene particles dispersed in the cured matrix formed by curing the RTV silicone rubber. The method for preparing the modified silicone rubber may comprise: preparing a polystyrene particle dispersion, wherein the polystyrene particle dispersion includes the polystyrene particles and a diluent; obtaining a mixture by mixing the polystyrene particle dispersion with the RTV silicone rubber, thereby dispersing the polystyrene particles in the cured matrix formed by curing the RTV silicone rubber; and obtaining the modified silicone rubber by adding a curing agent into the mixture for curing.

### CROSS REFERENCE TO RELATED APPLICATIONS

This application is a continuation application of International Application No. PCT/CN2022/143488, filed on Dec. 29, 2022, which claims priority of the Chinese Patent Application No. 202210521312.1, filed on May 13, 2022, the entire contents of which are hereby incorporated by reference.

### TECHNICAL FIELD

The present disclosure relates to the technical field of acoustic materials, and in particular to modified silicone rubbers and method for preparing thereof, acoustic permeable components, ultrasonic probes, and ultrasonic diagnostic devices.

### BACKGROUND

An ultrasonic probe (also referred to as an ultrasonic transducer) is an energy converter that converts acoustic signals to electrical signals. It is a key component of a medical ultrasonic diagnostic device. Ultrasound imaging is an ultrasonic diagnosis and treatment method based on the ultrasonic probe. The ultrasonic waves harmless to the human body are used as information carriers. The ultrasonic waves have different acoustic responses to different human tissues. Ultrasonic images of human tissues can be obtained by analyzing ultrasonic echo signals, which are widely used in clinical diagnosis and intraoperative observation because of the harmlessness and convenience.

The acoustic permeable component, as an acoustic lens, is located on the outermost layer of the ultrasonic probe. It is difficult for the current acoustic permeable materials to meet characteristics of low acoustic attenuation and acoustic reflection coefficient, good acoustic impedance matching characteristics and high hardness simultaneously. Thus, it is difficult to satisfy the requirements of high service life, and relatively good imaging sensitivity and imaging quality. Therefore, it is desirable to provide modified silicone rubbers and method for preparing thereof, acoustic permeable components, ultrasonic probes, and ultrasonic diagnostic devices, to satisfy the above requirements simultaneously.

### SUMMARY

One of the embodiments of the present disclosure provides modified silicone rubber, comprising: a cured matrix formed by curing a room temperature vulcanized (RTV) silicone rubber; and polystyrene particles dispersed in the cured matrix formed by curing the RTV silicone rubber.

One of the embodiments of the present disclosure further provides a method for preparing a modified silicone rubber, comprising: preparing a polystyrene particle dispersion, wherein the polystyrene particle dispersion may include polystyrene particles and a diluent; obtaining a mixture mixing the polystyrene particle dispersion with a room temperature vulcanized silicone rubber; and obtaining the modified silicone rubber by adding a curing agent into the mixture for curing, and thereby dispersing the polystyrene particles in the cured matrix formed by curing the RTV silicone rubber.

In some embodiments, the diluent may include at least one of a reactive diluent and a non-reactive diluent.

In some embodiments, when the diluent includes the non-reactive diluent, the method may further include: removing the non-reactive diluent from the mixture prior to adding the curing agent to the mixture for curing.

In some embodiments, based on 100 parts by weight of the RTV silicone rubber, the curing agent may be 10 parts by weight, the polystyrene particles may be within a range of 15-60 parts by weight, and the diluent may be within a range of 10-30 parts by weight.

In some embodiments, the cured matrix formed by curing the RTV silicone rubber may be within a range of 110-140 parts by weight; and the polystyrene particles may be within a range of 15-60 parts by weight.

In some embodiments, the cured matrix formed by curing the RTV silicone rubber may be within a range of 110-140 parts by weight; and the polystyrene particles may be within a range of 15-40 parts by weight.

In some embodiments, the cured matrix formed by curing the RTV silicone rubber may be within a range of 110-140 parts by weight; and the polystyrene particles may be within a range of 15-20 parts by weight.

In some embodiments, a polystyrene particle of the polystyrene particles may be spherical.

In some embodiments, a particle diameter coefficient of variation (CV) of a polystyrene particle of the polystyrene particles may be less than 3%.

In some embodiments, a particle diameter of a polystyrene particle of the polystyrene particles may be within a range of 1 μm-20 μm.

In some embodiments, a particle diameter of a polystyrene particle of the polystyrene particles may be within a range of 1 μm-10 μm.

In some embodiments, an acoustic attenuation coefficient of the modified silicone rubber at a frequency of 5 MHz may be less than 28 dB/cm.

In some embodiments, an acoustic impedance of the modified silicone rubber may be greater than 1.12 Mrayl.

In some embodiments, an acoustic reflection coefficient of the modified silicone rubber may be less than 2.1%.

One of the embodiments of this specification also provides a use of the modified silicone rubber as an acoustic permeable material.

One of the embodiments of the present disclosure further provides an acoustic permeable component. The acoustic permeable component may comprise the modified silicone rubber.

One of the embodiments of the present disclosure further provides an ultrasonic probe, comprising: a probe body; and the acoustic permeable component, wherein the acoustic permeable component may be arranged on a surface of the probe body.

### BRIEF DESCRIPTION OF THE DRAWINGS

The present disclosure will be further illustrated by way of exemplary embodiments, which will be described in detail with the accompanying drawings. These embodiments are non-limiting. In these embodiments, the same count indicates the same structure, wherein:

FIG. 1 is a schematic structural diagram illustrating an exemplary ultrasonic diagnostic device according to some embodiments of the present disclosure; and

FIG. 2 is a flowchart illustrating an exemplary method for preparing a modified silicone rubber according to some embodiments of the present disclosure.

### DETAILED DESCRIPTION

In order to illustrate the technical solutions related to the embodiments of the present disclosure, brief introduction of the drawings referred to in the description of the embodiments is provided below. Obviously, drawings described below are only some examples or embodiments of the present disclosure. Those having ordinary skills in the art, without further creative efforts, may apply the present disclosure to other similar scenarios according to these drawings. Unless stated otherwise or obvious from the context, the same reference numeral in the drawings refers to the same structure and operation.

As used in the disclosure and the appended claims, the singular forms “a,” “an,” and “the” include plural referents unless the content clearly dictates otherwise. In general, the terms “comprise” and “include” merely prompt to include steps and elements that have been clearly identified, and these steps and elements do not constitute an exclusive listing. The methods or the devices may also include other steps or elements.

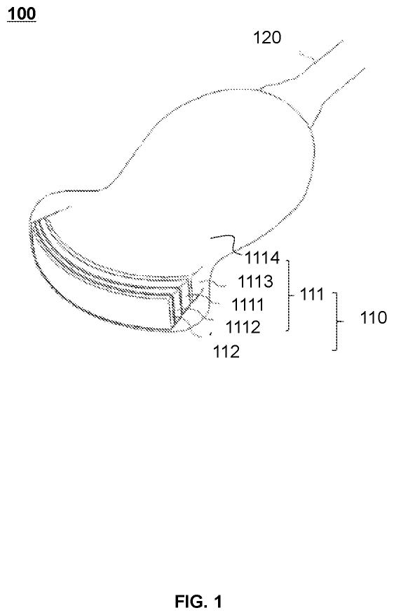

FIG. 1 is a schematic structural diagram illustrating an exemplary ultrasonic diagnostic device according to some embodiments of the present disclosure.

In some embodiments, as shown in FIG. 1, the ultrasonic diagnostic device 100 may comprise an ultrasonic probe 110 and a device host (not shown in the figures).

The ultrasonic probe 110 may transmit and receive ultrasonic waves, and perform an electro-acoustic signal converting process of the ultrasonic waves. The ultrasonic probe 110 may convert an electrical signal transmitted by the device host into a high-frequency oscillating ultrasonic signal, and may also convert the ultrasonic signal reflected from a diagnostic sample (hereinafter also referred to as an object), such as a tissue organ, into an electrical signal.

In some embodiments, the device host may be configured to process a signal received by the ultrasonic probe 110. In some embodiments, the device host may include a central processing unit (CPU), an application-specific integrated circuit (ASIC), an application-specific instruction set processor (ASIP), a graphics processor (GPU), a physical processing unit (PPU), a digital signal processor (DSP), a field programmable gate array (FPGA), a programmable logic device (PLD), a controller, a microcontroller unit, a reduced instruction set computer (RISC), a microprocessor, or the like, or any combination thereof.

In some embodiments, the ultrasonic diagnostic device 100 may include a display (not shown in the figures) for displaying information (e.g., sound wave information, image information, etc.) related to the object (e.g., a human tissue). In some embodiments, the display may include a liquid crystal display (LCD), a plasma display, a light emitting diode (LED) display, or the like, or any combination thereof.

In some embodiments, the ultrasonic diagnostic device 100 may include a cable 120 for connecting the ultrasonic probe 110, the device host, and the display.

In some embodiments, the ultrasonic probe 110, the device host, and/or the display of the ultrasonic diagnostic device 100 may communicate (e.g., data transmission) through a wireless network.

In some embodiments, as shown in FIG. 1, the ultrasonic probe 110 may include a probe body 111 and an acoustic permeable component 112.

In some embodiments, the probe body 111 may include a wafer 1111. The wafer 1111 may vibrate to generate ultrasonic waves by applying a voltage to electrodes at both ends of the wafer 1111. The ultrasonic waves emitted from the wafer 1111 may focus on the object (e.g., human tissue) through an acoustic lens. The ultrasonic waves transmitted from the object (e.g., human tissue) may carry information (e.g., information about the reflection, absorption, and scattering of sound waves) about the object (e.g., human tissue), and then converge on the wafer 1111 through the acoustic lens. The information may be received by the wafer 1111 and an electro-acoustic signal converting process may be performed on the information to obtain the electrical signal.

In some embodiments, the probe body 111 may include a matching layer 1112 located on a side of the wafer 1111 close to the object (e.g., human tissue). The matching layer 1112 may be configured to reduce a difference in acoustic impedance between the wafer 1111 and the object (e.g., human tissue), to realize efficient transmission and reception of the ultrasonic waves.

In some embodiments, the probe body 111 may include a sound-absorbing block 1113 located on a side of the wafer 1111 away from the matching layer 1112. The sound-absorbing block 1113 may reduce a pulse width of the ultrasonic waves by suppressing excessive vibration of the wafer 1111, to improve an axial resolution of an ultrasonic image.

In some embodiments, the probe body 111 may further include a support frame 1114 configured to support and protect the wafer 1111.

In some embodiments, as shown in FIG. 1, the sound-absorbing block 1113, the wafer 1111, and the matching layer 1112 may be sequentially disposed on the support frame 1114.

In some embodiments, the acoustic permeable component 112 may be disposed on a surface of the probe body 111 (e.g., matching layer 1112). The acoustic permeable component 112 may be in contact with the object (e.g., human tissue) when the ultrasonic probe 110 is configured to examine the object. In some embodiments, the acoustic permeable component 112 may include a modified silicone rubber.

In some embodiments, the modified silicone rubber may include a cured matrix formed by curing an RTV silicone rubber and polystyrene particles. The polystyrene particles may be dispersed in the cured matrix formed by curing RTV silicone rubber.

The RTV silicone rubber may be used as an acoustic permeable material, such as a molding material for the acoustic lens. Specifically, taking RTV615 silicone rubber as an example, an acoustic attenuation coefficient of the RTV615 silicone rubber at a frequency of 5 MHz (hereinafter also referred to as acoustic attenuation) is only 15.4 dB/cm, which has low attenuation characteristics. A sound velocity in the RTV615 silicone rubber is about ⅔ of that in a human soft tissue, and the acoustic attenuation coefficient is low. However, an acoustic impedance value of the RTV615 silicone rubber is 1.05 MRayl, which is quite different from an acoustic impedance value of the human body (1.5 MRayl). Therefore, the acoustic lens made of the RTV615 silicone rubber as the acoustic permeable material may have an obvious acoustic reflection signal at an interface with the human body, resulting in a decrease in the transmitted sound intensity, and the reflected sound waves may form artifacts (interference signals) during imaging, reducing imaging quality, and being unfavorable for ultrasonic imaging. In addition, a surface Shore hardness of the RTV615 silicone rubber may be 15.8, which is low in surface hardness and easily damaged.

In some embodiments of the present disclosure, the modified silicone rubber can be formed by modifying the RTV silicone rubber, to improve its acoustic matching characteristics, and make it have a relatively high surface hardness, relatively low acoustic attenuation, and acoustic reflection characteristics. In some embodiments, modifying the RTV silicone rubber may include dispersing the polystyrene particles in the cured matrix formed by curing the RTV silicone rubber.

The parts by weight of the cured matrix formed by curing the RTV silicone rubber and the polystyrene particles in the modified silicone rubber may affect the performance of the modified silicone rubber. For example, in the modified silicone rubber, if the parts by weight of the cured matrix formed by curing the RTV silicone rubber is too large or the parts by weight of the polystyrene particles is too small, the acoustic impedance of the modified silicone rubber may be small, which is different from that of the human body, and the hardness may be low. As another example, in the modified silicone rubber, if the parts by weight of the cured matrix formed by curing the RTV silicone rubber is too small or the parts by weight of the polystyrene particles is too large, the acoustic attenuation coefficient of the modified silicone rubber may increase too much. Therefore, in order to improve the performance (e.g., with good acoustic impedance matching, high hardness and small acoustic attenuation coefficient) of the modified silicone rubber, the parts by weight of the cured matrix formed by curing the RTV silicone rubber and the parts by weight of the polystyrene particles in the modified silicone rubber may need to meet preset conditions. The parts by weight may be presented by a count of the masses of different components in a mixture in units of mass. The number of parts by weight may indicate a mass relationship of different components in the mixture. The same parts by weight may represent the same mass.

In some embodiments, the cured matrix formed by curing the RTV silicone rubber in the modified silicone rubber may be within a range of 110-140 parts by weight, and the polystyrene particles may be within a range of 15-60 parts by weight. In some embodiments, the cured matrix formed by curing the RTV silicone rubber in the modified silicone rubber may be within a range of 110-140 parts by weight, and the polystyrene particles may be within a range of 15-55 parts by weight. In some embodiments, cured matrix formed by curing the RTV silicone rubber in the modified silicone rubber may be within a range of 110-140 parts by weight, and the polystyrene particles may be within a range of 15-50 parts by weight. In some embodiments, the cured matrix formed by curing the RTV silicone rubber in the modified silicone rubber may be within a range of 110-140 parts by weight, and the polystyrene particles may be within a range of 15-45 parts by weight. In some embodiments, cured matrix formed by curing the RTV silicone rubber in the modified silicone rubber may be within a range of 110-140 parts by weight, and the polystyrene particles may be within a range of 15-40 parts by weight. In some embodiments, the cured matrix formed by curing the RTV silicone rubber in the modified silicone rubber may be within a range of 110-140 parts by weight, and the polystyrene particles may be within a range of 15-35 parts by weight. In some embodiments, the cured matrix formed by curing the RTV silicone rubber in the modified silicone rubber may be within a range of 110-140 parts by weight, and the polystyrene particles may be within a range of 15-30 parts by weight. In some embodiments, the cured matrix formed by curing the RTV silicone rubber in the modified silicone rubber may be within a range of 110-140 parts by weight, and the polystyrene particles may be within a range of 15-25 parts by weight. In some embodiments, the cured matrix formed by curing the RTV silicone rubber in the modified silicone rubber may be within a range of 110-140 parts by weight, and the polystyrene particles may be within a range of 15-20 parts by weight. In some embodiments, the cured matrix formed by curing the RTV silicone rubber in the modified silicone rubber may be within a range of 110-140 parts by weight, and the polystyrene particles may be within a range of 15-18 parts by weight.

In some embodiments, the cured matrix formed by curing the RTV silicone rubber in the modified silicone rubber may be within a range of 110-140 parts by weight, and the polystyrene particles may be within a range of 20-60 parts by weight. In some embodiments, the cured matrix formed by curing the RTV silicone rubber in the modified silicone rubber may be within a range of 110-140 parts by weight, and the polystyrene particles may be within a range of 20-50 parts by weight. In some embodiments, the cured matrix formed by curing the RTV silicone rubber in the modified silicone rubber may be within a range of 110-140 parts by weight, and the polystyrene particles may be within a range of 20-40 parts by weight. In some embodiments, the cured matrix formed by curing the RTV silicone rubber in the modified silicone rubber may be within a range of 110-140 parts by weight, and the polystyrene particles may be within a range of 20-30 parts by weight. In some embodiments, the cured matrix formed by curing the RTV silicone rubber in the modified silicone rubber may be within a range of 110-140 parts by weight, and the polystyrene particles may be within a range of 30-60 parts by weight. In some embodiments, the cured matrix formed by curing the RTV silicone rubber in the modified silicone rubber may be within a range of 110-140 parts by weight, and the polystyrene particles may be within a range of 30-50 parts by weight. In some embodiments, the cured matrix formed by curing the RTV silicone rubber in the modified silicone rubber may be within a range of 110-140 parts by weight, and the polystyrene particles may be within a range of 30-40 parts by weight.

In some embodiments, the cured matrix formed by curing the RTV silicone rubber in the modified silicone rubber may be within a range of 110-135 parts by weight, and the polystyrene particles may be within a range of 15-60 parts by weight. In some embodiments, the cured matrix formed by curing the RTV silicone rubber in the modified silicone rubber may be within a range of 110-130 parts by weight, and the polystyrene particles may be within a range of 15-60 parts by weight. In some embodiments, the cured matrix formed by curing the RTV silicone rubber in the modified silicone rubber may be within a range of 110-125 parts by weight, and of the polystyrene particles may be within a range of 15-60 parts by weight. In some embodiments, the cured matrix formed by curing the RTV silicone rubber in the modified silicone rubber may be within a range of 110-120 parts by weight, and the polystyrene particles may be within a range of 15-60 parts by weight. In some embodiments, the cured matrix formed by curing the RTV silicone rubber in the modified silicone rubber may be within a range of 110-115 parts by weight, and the polystyrene particles may be within a range of 15-60 parts by weight.

In some embodiments, a polystyrene particle of the polystyrene particles may be spherical. In some embodiments, a polystyrene particle of the polystyrene particles may have a sheet structure. In some embodiments, a polystyrene particle of the polystyrene particles may also have other structures such as an ellipsoid structure, a prism structure, or a pyramid structure.

In some embodiments, the polystyrene particles may include various structures. For example, the polystyrene particles may include at least two of a spherical structure, the sheet structure, the ellipsoid structure, the prism structure, the pyramid structure, or the like. In some embodiments, the polystyrene particles may be monodisperse particles or particles containing one structure.

As described herein, when a polystyrene particle of the polystyrene particles is not spherical, the particle diameter of a polystyrene particle of the polystyrene particles may be expressed as an equivalent particle diameter. The particle diameter of a polystyrene particle of the polystyrene particles may affect the performance of the modified silicone rubber. For example, if the particle diameter of a polystyrene particle of the polystyrene particles is too large, a diffraction effect of the ultrasonic waves in the prepared modified silicone rubber may be poor, which in turn may cause the acoustic attenuation coefficient of the modified silicone rubber to increase too much. As another example, if the particle diameter of a polystyrene particle of the polystyrene particles is too small, a dispersion uniformity of the polystyrene particles in the RTV silicone rubber may be poor, resulting in a poor solid phase homogeneity of the modified silicone rubber, which may further cause the acoustic attenuation coefficient of the modified silicone rubber to increase too much. Therefore, in order to improve the performance of the modified silicone rubber (e.g., to have a small acoustic attenuation coefficient), the particle diameter of a polystyrene particle of the polystyrene particles may need to meet preset conditions.

In some embodiments, the particle diameters of the polystyrene particles dispersed in different modified silicone rubbers may be the same or different. The particle diameters of the polystyrene particles dispersed in different modified silicone rubbers may be within a range of 1 μm-20 μm. In some embodiments, the particle diameters of the polystyrene particles dispersed in different modified silicone rubbers may be within a range of 1 μm-18 μm. In some embodiments, the particle diameters of the polystyrene particles dispersed in different modified silicone rubbers may be within a range of 1 μm-16 μm. In some embodiments, the particle diameters of the polystyrene particles dispersed in different modified silicone rubbers may be within a range of 1 μm-14 μm. In some embodiments, the particle diameters of the polystyrene particles dispersed in different modified silicone rubbers may be within a range of 1 μm-12 μm. In some embodiments, the particle diameters of the polystyrene particles dispersed in different modified silicone rubbers may be within a range of 1 μm-10 μm. In some embodiments, the particle diameters of the polystyrene particles dispersed in different modified silicone rubbers may be within a range of 1 μm-8 μm. In some embodiments, the particle diameters of the polystyrene particles dispersed in different modified silicone rubbers may be within a range of 1 μm-6 μm. In some embodiments, the particle diameters of the polystyrene particles dispersed in different modified silicone rubbers may be within a range of 1 μm-4 μm. In some embodiments, the particle diameters of the polystyrene particles dispersed in different modified silicone rubbers may be within a range of 1 μm-2 μm.

In some embodiments, a particle diameter coefficient of variation (CV) may represent a width of a distribution of a particle diameter. A CV value may refer to a relative standard deviation, which may be expressed as a ratio of a standard deviation (SD) to an average value. Correspondingly, the particle diameter CV may be expressed as a ratio of a particle diameter SD to an average particle diameter.

The particle diameter CV of the polystyrene particles may affect the performance of the modified silicone rubber. For example, if the particle diameter CV of the polystyrene particles is too large, the solid phase homogeneity of the modified silicone rubber may be poor. Therefore, in order to improve the solid phase homogeneity of the prepared modified silicone rubber, further reduce a degree of scattering of ultrasonic waves thereof, and improve the acoustic impedance of the modified silicone rubber, the particle diameter CV of the polystyrene particles dispersed in the same modified silicone rubber may meet the preset conditions. That is, the particle diameters of different polystyrene particles dispersed in the same modified silicone rubber may meet certain requirements, so that the CV may meet the preset conditions (i.e., the particle diameters of the polystyrene particles dispersed in the same modified silicone rubber may be highly homogenized). For example, the particle diameters of different polystyrene particles dispersed in the same modified silicone rubber may be the same. For example, a difference between a largest particle diameter and a smallest particle diameter of different polystyrene particles dispersed in the same modified silicone rubber may be less than a certain threshold (e.g., 0.1 μm, 0.2 μm, 0.3 μm, 0.4 μm, or 0.5 μm, etc.).

In some embodiments, the particle diameter CV of the polystyrene particles dispersed in the same modified silicone rubber may be less than 3%. In some embodiments, the particle diameter CV of the polystyrene particles dispersed in the same modified silicone rubber may be less than 2.5%. In some embodiments, the particle diameter CV of the polystyrene particles dispersed in the same modified silicone rubber may be less than 2%. In some embodiments, the particle diameter CV of the polystyrene particles dispersed in the same modified silicone rubber may be less than 1.5%. In some embodiments, the particle diameter CV of the polystyrene particles dispersed in the same modified silicone rubber may be less than 1%. In some embodiments, the particle diameter CV of the polystyrene particles dispersed in the same modified silicone rubber may be less than 0.5%. In some embodiments, the particle diameter CV of the polystyrene particles dispersed in the same modified silicone rubber may be less than 0.3%. In some embodiments, the particle diameter CV of the polystyrene particles dispersed in the same modified silicone rubber may be less than 1%.

In some embodiments, the particle diameter of a polystyrene particle of the polystyrene particles dispersed in the same modified silicone rubber may be within a range of 0.8 μm-1.2 μm. In some embodiments, the particle diameter of the polystyrene particle of the polystyrene particles dispersed in the same modified silicone rubber may be within a range of 0.9 μm-1.1 μm. In some embodiments, the particle diameter of the polystyrene particle of the polystyrene particles dispersed in the same modified silicone rubber may be within a range of 1 μm±0.1 μm. In some embodiments, the particle diameter of the polystyrene particle of the polystyrene particles dispersed in the same modified silicone rubber may be within a range of 1 μm±0.2 μm.

In some embodiments, the particle diameter of the polystyrene particle of the polystyrene particles dispersed in the same modified silicone rubber may be within a range of 9.8 μm-10.2 μm. In some embodiments, the particle diameter of the polystyrene particle of the polystyrene particles dispersed in the same modified silicone rubber may be within a range of 9.9 μm-10.1 μm. In some embodiments, the particle diameter of the polystyrene particle of the polystyrene particles dispersed in the same modified silicone rubber may be within a range of 10 μm±0.1 μm. In some embodiments, the particle diameter of the polystyrene particle of the polystyrene particles dispersed in the same modified silicone rubber may be within a range of 10 μm±0.2 μm.

In some embodiments, the particle diameter of the polystyrene particle of the polystyrene particles dispersed in the same modified silicone rubber may be within a range of 19.8 μm-20.2 μm. In some embodiments, the particle diameter of the polystyrene particle of the polystyrene particles dispersed in the same modified silicone rubber may be within a range of 19.9 μm-20.1 μm. In some embodiments, the particle diameter of the polystyrene particle of the polystyrene particles dispersed in the same modified silicone rubber may be within a range of 20 μm±0.1 μm. In some embodiments, the particle diameter of the polystyrene particle of the polystyrene particles dispersed in the same modified silicone rubber may be within a range of 20 μm±0.2 μm.

The polystyrene particles may not contain polar functional groups. The polystyrene particles may not agglomerate. The polystyrene particles may have good dispersibility. Under the action of high-speed stirring, the polystyrene particles may be uniformly dispersed in the RTV silicone rubber, which may avoid the problem of strong reflection of sound waves caused by the agglomeration of the polystyrene particles, and may make the modified silicone rubber have a low acoustic reflection coefficient.

A density of the polystyrene particles may be 1.05 g/cm³, which may be almost the same as a density of the RTV silicone rubber. The polystyrene particles may be used as modified fillers of the RTV silicone rubber. The polystyrene particles may not undergo phase separation of floating and sedimentation in the RTV silicone rubber, and may have good compatibility with the RTV silicone rubber, thereby ensuring that the cured polystyrene particles may be uniformly dispersed in the cured matrix formed by curing the RTV silicone rubber.

The acoustic impedance of the polystyrene particles may be approximately 2.5 MRayl, and the acoustic attenuation of the polystyrene particles at an acoustic frequency of 5 MHz may be only 1.7 dB/cm. The polystyrene particles may have relatively high acoustic impedance and relatively low acoustic attenuation characteristics. The polystyrene particles with a high degree of homogeneity in particle diameter may be used. For example, the particle diameter CV of a polystyrene particle of the polystyrene particles may be less than 3%, and at a composition ratio of the polystyrene particles with the cured matrix formed by curing the RTV silicone rubber may be coordinated, to make the polystyrene particles fill in the cured matrix formed by curing the RTV silicone rubber, thereby realizing excellent solid phase homogeneity and low degree of scattering of ultrasonic waves. Compared with a cured matrix formed by curing an unmodified silicone rubber, the polystyrene particles may increase the acoustic impedance of the prepared modified silicone rubber, thereby improving the acoustic matching characteristics of the modified silicone rubber with the human tissue, reducing the acoustic reflection coefficient, improving the surface hardness of the modified silicone rubber, and further ensuring the low acoustic attenuation characteristics.

According to the embodiments of the present disclosure, the modified silicone rubber may have a low acoustic attenuation, a low acoustic reflection coefficient, good acoustic impedance matching characteristics, and high hardness. The modified silicone rubber may be used as the acoustic permeable materials for the ultrasonic diagnostic device. The modified silicone rubber may have high service life, improve the imaging sensitivity and the imaging quality of the ultrasonic diagnostic device, and have relatively good practicability.

In some embodiments, the acoustic attenuation coefficient of the modified silicone rubber at a frequency of 5 MHz may be less than 28 dB/cm. In some embodiments, the acoustic attenuation coefficient of the modified silicone rubber at the frequency of 5 MHz may be less than or equal to 26 dB/cm. In some embodiments, the acoustic attenuation coefficient of the modified silicone rubber at the frequency of 5 MHz may be less than or equal to 24 dB/cm. In some embodiments, the acoustic attenuation coefficient of the modified silicone rubber at the frequency of 5 MHz may be less than or equal to 22 dB/cm. In some embodiments, the acoustic attenuation coefficient of the modified silicone rubber at the frequency of 5 MHz may be less than or equal to 20 dB/cm. In some embodiments, the acoustic attenuation coefficient of the modified silicone rubber at the frequency of 5 MHz may be less than or equal to 18 dB/cm. In some embodiments, the acoustic attenuation coefficient of the modified silicone rubber at the frequency of 5 MHz may be less than or equal to 16 dB/cm. In some embodiments, the acoustic attenuation coefficient of the modified silicone rubber at the frequency of 5 MHz may be less than or equal to 14 dB/cm. In some embodiments, the acoustic attenuation coefficient of the modified silicone rubber at the frequency of 5 MHz may be less than or equal to 12 dB/cm. In some embodiments, the acoustic attenuation coefficient of the modified silicone rubber at the frequency of 5 MHz may be less than or equal to 10 dB/cm.

In some embodiments, compared with the cured matrix formed by curing the RTV silicone rubber, an increase rate of the acoustic attenuation coefficient of the modified silicone rubber may be less than or equal to 75% at a same frequency. In some embodiments, compared with the cured matrix formed by curing the RTV silicone rubber, the increase rate of the acoustic attenuation coefficient of the modified silicone rubber may be less than or equal to 70% at the same frequency. In some embodiments, compared with the cured matrix formed by curing the RTV silicone rubber, the increase rate of the acoustic attenuation coefficient of the modified silicone rubber may be less than or equal to 65% at the same frequency. In some embodiments, compared with the cured matrix formed by curing the RTV silicone rubber, the increase rate of the acoustic attenuation coefficient of the modified silicone rubber may be less than or equal to 60% at the same frequency. In some embodiments, compared with the cured matrix formed by curing the RTV silicone rubber, the increase rate of the acoustic attenuation coefficient of the modified silicone rubber may be less than or equal to 55% at the same frequency. In some embodiments, compared with the cured matrix formed by curing the RTV silicone rubber, the increase rate of the acoustic attenuation coefficient of the modified silicone rubber may be less than or equal to 50% at the same frequency. In some embodiments, compared with the cured matrix formed by curing the RTV silicone rubber, the increase rate of the acoustic attenuation coefficient of the modified silicone rubber may be less than or equal to 45% at the same frequency. In some embodiments, compared with the cured matrix formed by curing the RTV silicone rubber, the increase rate of the acoustic attenuation coefficient of the modified silicone rubber may be less than or equal to 40% at the same frequency. In some embodiments, compared with the cured matrix formed by curing the RTV silicone rubber, the increase rate of the acoustic attenuation coefficient of the modified silicone rubber may be less than or equal to 35% at the same frequency. In some embodiments, compared with the cured matrix formed by curing the RTV silicone rubber, the increase rate of the acoustic attenuation coefficient of the modified silicone rubber may be less than or equal to 30% at the same frequency. In some embodiments, compared with the cured matrix formed by curing the RTV silicone rubber, the increase rate of the acoustic attenuation coefficient of the modified silicone rubber may be less than or equal to 25% at the same frequency. In some embodiments, compared with the cured matrix formed by curing the RTV silicone rubber, the increase rate of the acoustic attenuation coefficient of the modified silicone rubber may be less than or equal to 20% at the same frequency. In some embodiments, compared with the cured matrix formed by curing the RTV silicone rubber, the increase rate of the acoustic attenuation coefficient of the modified silicone rubber may be less than or equal to 15% at the same frequency. In some embodiments, compared with the cured matrix formed by curing the RTV silicone rubber, the increase rate of the acoustic attenuation coefficient of the modified silicone rubber may be less than or equal to 10% at the same frequency. In some embodiments, compared with the cured matrix formed by curing the RTV silicone rubber, the increase rate of the acoustic attenuation coefficient of the modified silicone rubber may be less than or equal to 5% at the same frequency.

In some embodiments, the acoustic impedance of the modified silicone rubber may be within a range of 1.1 Mrayl-1.5 Mrayl. In some embodiments, the acoustic impedance of the modified silicone rubber may be in the range of 1.15 Mrayl-1.45 Mrayl. In some embodiments, the acoustic impedance of the modified silicone rubber may be within a range of 1.2 Mrayl-1.4 Mrayl. In some embodiments, the acoustic impedance of the modified silicone rubber may be within a range of 1.25 Mrayl-1.35 Mrayl. In some embodiments, the acoustic impedance of the modified silicone rubber may be within a range of 1.2 Mrayl-1.5 Mrayl. In some embodiments, the acoustic impedance of the modified silicone rubber may be within a range of 1.3 Mrayl-1.5 Mrayl. In some embodiments, the acoustic impedance of the modified silicone rubber may be within a range of 1.4 Mrayl-1.5 Mrayl.

In some embodiments, the acoustic impedance of the modified silicone rubber may be greater than 1.12 Mrayl. In some embodiments, the acoustic impedance of the modified silicone rubber may be greater than 1.15 Mrayl. In some embodiments, the acoustic impedance of the modified silicone rubber may be greater than 1.17 Mrayl. In some embodiments, the acoustic impedance of the modified silicone rubber may be greater than 1.2 Mrayl. In some embodiments, the acoustic impedance of the modified silicone rubber may be greater than 1.25 Mrayl. In some embodiments, the acoustic impedance of the modified silicone rubber may be greater than 1.3 Mrayl. In some embodiments, the acoustic impedance of the modified silicone rubber may be greater than 1.35 Mrayl.

In some embodiments, compared with the cured matrix formed by curing the RTV silicone rubber, an increase rate of the acoustic impedance of the modified silicone rubber may be within a range of 7%-43%. In some embodiments, compared with the cured matrix formed by curing the RTV silicone rubber, the increase rate of the acoustic impedance of the modified silicone rubber may be within a range of 7%-40%. In some embodiments, compared with the cured matrix formed by curing the RTV silicone rubber, the increase rate of the acoustic impedance of the modified silicone rubber may be within a range of 7%-35%. In some embodiments, compared with the cured matrix formed by curing the RTV silicone rubber, the increase rate of the acoustic impedance of the modified silicone rubber may be within a range of 7%-30%. In some embodiments, compared with the cured matrix formed by curing the RTV silicone rubber, the increase rate of the acoustic impedance of the modified silicone rubber may be within a range of 7%-25%. In some embodiments, compared with the cured matrix formed by curing the RTV silicone rubber, the increase rate of the acoustic impedance of the modified silicone rubber may be within a range of 7%-20%. In some embodiments, compared with the cured matrix formed by curing the RTV silicone rubber, the increase rate of the acoustic impedance of the modified silicone rubber may be within a range of 7%-15%.

In some embodiments, compared with the cured matrix formed by curing the RTV silicone rubber, the increase rate of the acoustic impedance of the modified silicone rubber may be within a range of 5.6%-40%. In some embodiments, compared with the cured matrix formed by curing the RTV silicone rubber, the increase rate of the acoustic impedance of the modified silicone rubber may be within a range of 8%-40%. In some embodiments, compared with the cured matrix formed by curing the RTV silicone rubber, the increase rate of the acoustic impedance of the modified silicone rubber may be within a range of 10%-40%. In some embodiments, compared with the cured matrix formed by curing the RTV silicone rubber, the increase rate of the acoustic impedance of the modified silicone rubber may be within a range of 15%-40%. In some embodiments, compared with the cured matrix formed by curing the RTV silicone rubber, the increase rate of the acoustic impedance of the modified silicone rubber may be within a range of 20%-40%. In some embodiments, compared with the cured matrix formed by curing the RTV silicone rubber, the increase rate of the acoustic impedance of the modified silicone rubber may be within a range of 25%-40%. In some embodiments, compared with the cured matrix formed by curing the RTV silicone rubber, the increase rate of the acoustic impedance of the modified silicone rubber may be within a range of 30%-40%. In some embodiments, compared with the cured matrix formed by curing the RTV silicone rubber, the increase rate of the acoustic impedance of the modified silicone rubber may be within a range of 35%-40%.

In some embodiments, a difference between the acoustic impedance of the modified silicone rubber and the acoustic impedance of the human tissue may be within a range of 0.1 MRayl-0.4 MRayl. In some embodiments, the difference between the acoustic impedance of the modified silicone rubber and the acoustic impedance of the human tissue may be within a range of 0.15 MRayl-0.35 MRayl. In some embodiments, the difference between the acoustic impedance of the modified silicone rubber and the acoustic impedance of the human tissue may be within a range of 0.2 MRayl-0.3 MRayl. In some embodiments, the difference between the acoustic impedance of the modified silicone rubber and the acoustic impedance of the human tissue may be within a range of 0.24 MRayl-0.3 MRayl. In some embodiments, the difference between the acoustic impedance of the modified silicone rubber and the acoustic impedance of the human tissue may be within a range of 0.1 MRayl-0.3 MRayl. In some embodiments, the difference between the acoustic impedance of the modified silicone rubber and the acoustic impedance of the human tissue may be within a range of 0.1 MRayl-0.2 MRayl. For example, when the acoustic impedance of the human tissue is 1.5 MRayl, the acoustic impedance of the modified silicone rubber may be 1.1 MRayl, or 1.2 MRayl, or 1.3 MRayl, or 1.4 MRayl.

In some embodiments, a Shore hardness of the modified silicone rubber may be within a range of 17-60. In some embodiments, the Shore hardness of the modified silicone rubber may be within a range of 17-50. In some embodiments, the Shore hardness of the modified silicone rubber may be within a range of 17-40. In some embodiments, the Shore hardness of the modified silicone rubber may be within a range of 17-30. In some embodiments, the Shore hardness of the modified silicone rubber may be within a range of 17-25. In some embodiments, the Shore hardness of the modified silicone rubber may be within a range of 20-60. In some embodiments, the Shore hardness of the modified silicone rubber may be within a range of 30-60. In some embodiments, the Shore hardness of the modified silicone rubber may be within a range of 40-60. In some embodiments, the Shore hardness of the modified silicone rubber may be within a range of 50-60.

In some embodiments, compared with the cured matrix formed by curing the RTV silicone rubber, an increase rate of the Shore hardness of the modified silicone rubber may be within a range of 9.4%-150% In some embodiments, compared with the cured matrix formed by curing the RTV silicone rubber, the increase rate of the Shore hardness of the modified silicone rubber may be within a range of 10%-150%. In some embodiments, compared with the cured matrix formed by curing the RTV silicone rubber, the increase rate of the Shore hardness of the modified silicone rubber may be within a range of 20%-150%. In some embodiments, compared with the cured matrix formed by curing the RTV silicone rubber, the increase rate of the Shore hardness of the modified silicone rubber may be within a range of 30%-150%. In some embodiments, compared with the cured matrix formed by curing the RTV silicone rubber, the increase rate of the Shore hardness of the modified silicone rubber may be within a range of 40%-150%. In some embodiments, compared with the cured matrix formed by curing the RTV silicone rubber, the increase rate of the Shore hardness of the modified silicone rubber may be within a range of 50%-150%. In some embodiments, compared with the cured matrix formed by curing the RTV silicone rubber, the increase rate of the Shore hardness of the modified silicone rubber may be within a range of 60%-150%. In some embodiments, compared with the cured matrix formed by curing the RTV silicone rubber, the increase rate of the Shore hardness of the modified silicone rubber may be within a range of 70%-150%. In some embodiments, compared with the cured matrix formed by curing the RTV silicone rubber, the increase rate of the Shore hardness of the modified silicone rubber may be within a range of 80%-150%. In some embodiments, compared with the cured matrix formed by curing the RTV silicone rubber, the increase rate of the Shore hardness of the modified silicone rubber may be within a range of 90%-150%. In some embodiments, compared with the cured matrix formed by curing the RTV silicone rubber, the increase rate of the Shore hardness of the modified silicone rubber may be within a range of 100%-150%. In some embodiments, compared with the cured matrix formed by curing the RTV silicone rubber, the increase rate of the Shore hardness of the modified silicone rubber may be within a range of 110%-150%. In some embodiments, compared with the cured matrix formed by curing the RTV silicone rubber, the increase rate of the Shore hardness of the modified silicone rubber may be within a range of 120%-150%. In some embodiments, compared with the cured matrix formed by curing the RTV silicone rubber, the increase rate of the Shore hardness of the modified silicone rubber may be within a range of 130%-150%. In some embodiments, compared with the cured matrix formed by curing the RTV silicone rubber, the increase rate of the Shore hardness of the modified silicone rubber may be within a range of 140%-150%.

In some embodiments, the acoustic reflection coefficient of the modified silicone rubber may be less than 2.1%. In some embodiments, the acoustic reflection coefficient of the modified silicone rubber may be less than or equal to 2%. In some embodiments, the acoustic reflection coefficient of the modified silicone rubber may be less than or equal to 1.8%. In some embodiments, the acoustic reflection coefficient of the modified silicone rubber may be less than or equal to 1.6%. In some embodiments, the acoustic reflection coefficient of the modified silicone rubber may be less than or equal to 1.4%. In some embodiments, the acoustic reflection coefficient of the modified silicone rubber may be less than or equal to 1.2%. In some embodiments, the acoustic reflection coefficient of the modified silicone rubber may be less than or equal to 1.0%. In some embodiments, the acoustic reflection coefficient of the modified silicone rubber may be less than or equal to 0.8%. In some embodiments, the acoustic reflection coefficient of the modified silicone rubber may be less than or equal to 0.6%. In some embodiments, the acoustic reflection coefficient of the modified silicone rubber may be less than or equal to 0.5%. In some embodiments, the acoustic reflection coefficient of the modified silicone rubber may be less than or equal to 0.4%. In some embodiments, the acoustic reflection coefficient of the modified silicone rubber may be less than or equal to 0.2%.

It should be noted that the above description is for illustration and description purposes only, and does not limit the scope of application of the present disclosure. Various modifications and variations may be made by those skilled in the art under the guidance of the present disclosure. However, such modifications and variations remain within the scope of the present disclosure.

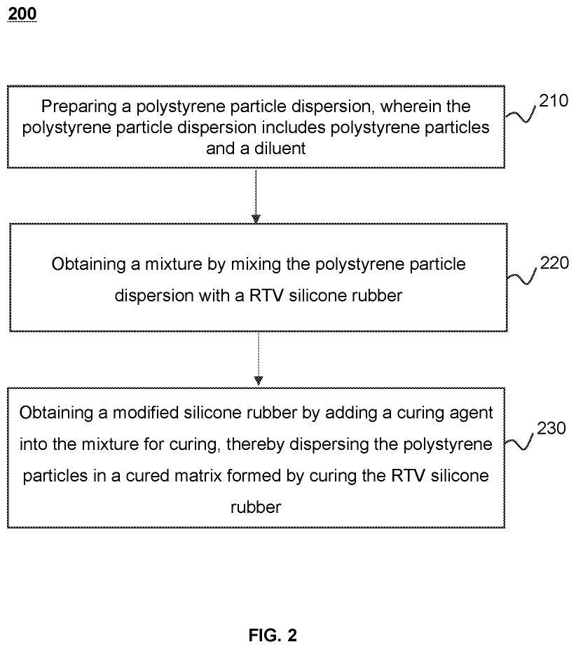

FIG. 2 is a flowchart illustrating an exemplary method for preparing a modified silicone rubber according to some embodiments of the present disclosure.

In some embodiments, a process 200 may be performed by a processing logic. The processing logic may include hardware (e.g., a circuit, a dedicated logic, a programmable logic, a microcode, etc.), software (e.g., an instruction running on a processing device to perform hardware emulation), or the like, or any combination thereof. One or more operations in the process 200 of the method for preparing the modified silicone rubber in FIG. 2 may be implemented by the processing device. For example, the process 200 may be stored in a storage device in the form of instructions, and invoked and/or performed by the processing device.

As shown in FIG. 2, the method for preparing the modified silicone rubber may comprise the following operations.

In 210, a polystyrene particle dispersion may be prepared.

In some embodiments, the polystyrene particle dispersion may include polystyrene particles and a diluent. Descriptions regarding the polystyrene particles may be found elsewhere of the present disclosure (e.g., FIG. 1 and related descriptions thereof), which is not repeated herein.

The diluents may be configured to disperse the polystyrene particles. In some embodiments, the diluent may include at least one of a reactive diluent and a non-reactive diluent. In some embodiments, the diluent may all be the reactive diluent, or the non-reactive diluent. In some embodiments, the diluent may be a combination of the reactive diluent and the non-reactive diluent.

In some embodiments, the reactive diluent may participate in a reaction (e.g., a curing reaction). For example, a cured matrix formed by curing an RTV silicone rubber may be formed by curing the RTV silicone rubber, a curing agent, and the reactive diluent, thereby increasing a crosslinking density of the cured matrix formed by curing the RTV silicone rubber. A hardness of the prepared modified silicone rubber may be improved. In some embodiments, a mass of the cured matrix formed by curing the RTV silicone rubber may be theoretically equal to a sum of masses of the added RTV silicone rubber, the curing agent, and the reactive diluent. In some embodiments, the reactive diluent may include silicone oil such as methyl silicone oil and simethicone, or substituted silicone oils such as methyl silicone oil and simethicone. In some embodiments, substituent groups may include halogens, alkanes, arenes, or the like, such as fluorine-substituted methyl silicone oil, and fluorine-substituted simethicone.

In some embodiments, the non-reactive diluent may not participate in the reaction (e.g., the curing reaction). In some embodiments, the non-reactive diluent may include ethanol, toluene, or the like.

In some embodiments, the polystyrene particle dispersion may be prepared by mixing the polystyrene particles with the diluent. In some embodiments, a mixing method may include, but is not limited to, mechanical stirring, shaking, or the like.

In 220, a mixture may be obtained by mixing the polystyrene particle dispersion with the RTV silicone rubber.

Descriptions regarding the RTV silicone rubber may be found elsewhere of the present disclosure (e.g., FIG. 1 and related descriptions thereof), which is not repeated herein.

In some embodiments, the mixture may be obtained by adding the polystyrene particle dispersion in small quantities into the RTV silicone rubber for multiple times and mixed, thereby improving a dispersion uniformity of the polystyrene particles in the RTV silicone rubber. For example, the polystyrene particle dispersion may be divided into four parts to be added to the RTV silicone rubber for mixing in turn, and then stirred to disperse uniformly. In some embodiments, the mixing method may include, but is not limited to, mechanical stirring, shaking, or the like.

In step 230, the modified silicone rubber may be obtained by adding the curing agent into the mixture for curing, thereby dispersing the polystyrene particles in the cured matrix formed by curing the RTV silicone rubber.

In some embodiments, the curing agent may cause the RTV silicone rubber to undergo the curing reaction to form the cured matrix obtained by curing the RTV silicone rubber. In some embodiments, the curing agent may be determined based on the RTV silicone rubber. In some embodiments, the curing treatment may be performed at room temperature (20° C.-35° C.). In some embodiments, a curing treatment time may be within a range of 36 h-60 h. In some embodiments, the curing treatment time may be within a range of 40 h-55 h. In some embodiments, the curing treatment time may be within a range of 45 h-50 h. In some embodiments, the curing treatment time may be within a range of 45 h-48 h. In some embodiments, the curing treatment time may be 36 h, or 40 h, or 45 h, or 48 h, or 50 h, or 55 h, or 60 h.

In some embodiments, when the diluent includes the non-reactive diluent, prior to adding the curing agent to the mixture for curing, the method may further include: removing the non-reactive diluent from the mixture.

In some embodiments, the non-reactive diluent may be removed from the mixture by heating. In some embodiments, a heating temperature may be determined based on a type of the non-reactive diluent. In some embodiments, the heating temperature may be within a range of 50° C.-80° C. For example, the heating temperature may be 50° C., 60° C., 70° C. or 80° C. In some embodiments, a heating time may be within a range of 1 h-4 h. For example, the heating time may be 1 h, 2 h, 3 h or 4 h. In some embodiments, when the non-reactive diluent is ethanol, the heating temperature for removing the ethanol may be 50° C., and the heating time may be 2 h. In some embodiments, the heating treatment may be performed in a heating device such as an oven.

When the diluent is the non-reactive diluent, a mass of the cured matrix formed by curing the RTV silicone rubber may be theoretically equal to a sum of masses of the added RTV silicone rubber and the curing agent.

In some embodiments, preparation raw materials of the method for preparing the modified silicon rubber may include, in parts by weight, based on 100 parts by weight of RTV silicone rubber, 10-40 parts by weight of the curing agent, 15-60 parts by weight of the polystyrene particles, and 10-30 parts by weight of the diluent.

In some embodiments, based on 100 parts by weight of the RTV silicone rubber, the curing agent may be within a range of 10-40 parts by weight. In some embodiments, the curing agent may be within a range of 10-35 parts by weight. In some embodiments, the curing agent may be within a range of 10-30 parts by weight. In some embodiments, the curing agent may be within a range of 10-25 parts by weight. In some embodiments, the curing agent may be within a range of 10-20 parts by weight. In some embodiments, the curing agent may be within a range of 10-15 parts by weight. In some embodiments, based on 100 parts by weight of the RTV silicone rubber, the curing agent may be 10, or 15, or 20, or 25, or 30, or 35, or 40 parts by weight.

In some embodiments, based on 100 parts by weight of the RTV silicone rubber, the polystyrene particles may be within a range of 15-60 parts by weight. In some embodiments, the polystyrene particles may be within a range of 15-55 parts by weight. In some embodiments, the polystyrene particles may be within a range of 15-50 parts by weight. In some embodiments, the polystyrene particles may be within a range of 15-45 parts by weight. In some embodiments, the polystyrene particles may be within a range of 15-40 parts by weight. In some embodiments, the polystyrene particles may be within a range of 15-35 parts by weight. In some embodiments, of the polystyrene particles may be within a range of 15-30 parts by weight. In some embodiments, the polystyrene particles may be within a range of 15-25 parts by weight. In some embodiments, the polystyrene particles may be within a range of 15-20 parts by weight. In some embodiments, the polystyrene particles may be within a range of 15-18 parts by weight. In some embodiments, the polystyrene particles may be within a range of 20-60 parts by weight. In some embodiments, the polystyrene particles may be within a range of 20-50 parts by weight. In some embodiments, the polystyrene particles may be within a range of 20-40 parts by weight. In some embodiments, the polystyrene particles may be within a range of 20-30 parts by weight. In some embodiments, based on 100 parts by weight of RTV silicone rubber, the polystyrene particles may be 15, 18, 20, 25, 30, 35, 40, 45, 50, 55, or 60 parts by weight.

In some embodiments, based on 100 parts by weight of the RTV silicone rubber, the diluent may be within a range of 10-30 parts by weight. In some embodiments, the diluent may be within a range of 10-25 parts by weight. In some embodiments, the diluent may be within a range of 10-20 parts by weight. In some embodiments, the diluent may be within a range of 10-15 parts by weight. In some embodiments, based on 100 parts by weight of the RTV silicone rubber, the diluent may be 10, 15, 20, 25, or 30 parts by weight.

In some embodiments, the method for preparing the modified silicone rubber may include: uniformly mixing the RTV silicone rubber, the polystyrene particles, and the diluent, and then obtaining the modified silicone rubber by adding the curing agent into the mixture for curing, thereby dispersing the polystyrene particles in the cured matrix formed by curing the RTV silicone rubber.

In some embodiments, descriptions regarding the parts by weight of the cured matrix formed by curing the RTV silicone rubber and the polystyrene particles in the modified silicone rubber may be found elsewhere of the present disclosure (e.g., FIG. 1 and related descriptions thereof), which is not repeated herein.

The method for preparing the modified silicone rubber provided in the embodiments of the present disclosure has a simple process, and the curing treatment may be carried out at room temperature, which is convenient for industrial production and has great practical application value.

It should be noted that the above description is for illustration and description purposes only, and does not limit the scope of application of the present disclosure. Various modifications and variations may be made by those skilled in the art under the guidance of the present disclosure. However, such modifications and variations remain within the scope of the present disclosure.

Some embodiments of the present disclosure further provide a use of the modified silicone rubber as an acoustic permeable material. For example, the modified silicone rubber may be used as the acoustic permeable material for an ultrasonic diagnostic device. As another example, the modified silicone rubber may be used as the acoustic permeable material of a transducer.

Through the co-action of the polystyrene particles and various components (e.g., the diluent), the modified silicone rubber obtained after the modification of the RTV silicone rubber has significantly improved acoustic matching characteristics with the human tissue, the surface hardness of the modified silicone rubber is significantly improved, and the acoustic attenuation characteristics may be kept at a low level, such that the modified silicone rubber is very suitable as the acoustic permeable material, for example, as a material for making acoustic permeable components such as an acoustic lens.

Some embodiments of the present disclosure further provide a use of the modified silicone rubber in preparation of the acoustic permeable component. For example, the modified silicone rubber may be used to prepare the acoustic permeable component of an ultrasonic probe of the ultrasonic diagnostic device. As another example, the modified silicone rubber may be used to prepare the acoustic permeable component of the transducer.

Some embodiments of the present disclosure further provide an acoustic permeable component comprising the modified silicone rubber.

In some embodiments, the acoustic permeable component may be an acoustic lens.

In some embodiments, the acoustic permeable component may be made of the modified silicone rubber. In some embodiments, the acoustic permeable component may include the modified silicone rubber, and may also include other components.

In some embodiments, the acoustic permeable component may be directly molded in a mold using the preparation raw materials for the modified silicone rubber, and then further processed as required.

The acoustic permeable component provided by the embodiment of present disclosure has a relatively large surface hardness. Thus, a service life may be effectively increased, acoustic matching characteristics with the human tissue may be improved, and the acoustic attenuation characteristics may be kept at a low level, such that the acoustic reflection signal at the interface between the acoustic permeable component and the human body may be reduced under a low effect of the acoustic attenuation, and the transmitted sound intensity can be increased, thereby effectively reducing artifacts (interference signals) and improving the imaging quality.

Some embodiments of the present disclosure further provide an ultrasonic probe comprising the acoustic permeable component, which may effectively improve the service life, reduce the acoustic reflection of the interface between the ultrasonic probe and the human body under a low effect of the acoustic attenuation, and improve the transmitted sound intensity, thereby effectively reducing artifacts (interfering signals) and improving the ultrasonic imaging quality.

Some embodiments of the present disclosure further provide an ultrasonic diagnostic device comprising a device host and the ultrasonic probe.

In order to make the purpose, technical solutions and advantages of the present disclosure concise and clear, the present disclosure is described with the following specific examples, but the present disclosure is by no means limited to these examples. The embodiments described below are only preferred embodiments of the present disclosure, which may be used to describe the present disclosure, and should not be construed as limiting the scope of the present disclosure. It should be noted that any modifications, equivalent substitutions and improvements made within the spirit and principles of the present disclosure shall be included within the protection scope of the present disclosure.

In order to better illustrate the present disclosure, the content of the present disclosure will be further described below in conjunction with the examples. The following are specific examples. The polystyrene particles with a particle diameter of 0.1 μm in the various examples and comparative examples were produced from Vmicro Nano's PST-100, the polystyrene particles with a particle diameter of 1 μm were produced from Vmicro Nano's PST 001UM, the polystyrene particles with a particle diameter of 10 μm were produced from PST 010UM of Vmicro Nano, and the polystyrene particles with a particle diameter of 20 μm were produced from PST 020UM of Vmicro Nano. The particle diameter CV of a polystyrene particle of the polystyrene particles of each particle diameter may be less than 3%. The manufacturer's model of the curing agent is Momentive 9482. The RTV silicone rubber may be Momentive RTV615.

### Example 1

At 25° C., 100 g RTV615 was added into a 250 mL flask. 10 g ethanol was used as a diluent, and stirred with 20 g polystyrene particles at 25° C. for 10 min, and a polystyrene particle dispersion was obtained after uniform mixing. A polystyrene particle of the polystyrene particles was spherical with a particle diameter of 1 μm. The polystyrene particle dispersion was added into the flask in four times, each time a quarter of the polystyrene particle dispersion was added, and stirred for 10 min. After the polystyrene particle dispersion is added, dried in an oven at 50° C. for 2 h, an ethanol solvent was removed, 10 g curing agent (hereinafter Momentive 9482) was added and stirred for 10 min, then poured into a mold and placed in a 50% RH constant temperature and humidity box at 25° C. for curing 48 h to obtain the modified silicone rubber. The modified silicone rubber may be used as an acoustic lens material.

### Example 2

At 25° C., 100 g RTV615 was added into a 250 mL flask. 10 g ethanol was used as a diluent, and stirred with 20 g polystyrene particles at 25° C. for 10 min, and a polystyrene particle dispersion was obtained after uniform mixing. A polystyrene particle of the polystyrene particles was spherical with a particle diameter of 10 μm. The polystyrene particle dispersion is added into the flask in four times, each time a quarter of the polystyrene particle dispersion was added, and stirred for 10 min. After the polystyrene particle dispersion was added, dried in an oven at 50° C. for 2 h, an ethanol solvent was removed, 10 g curing agent was added and stirred for 10 min, then poured into a mold, and placed in a 50% RH constant temperature and humidity box at 25° C. for curing 48 h to obtain modified silicone rubber. The modified silicone rubber may be used as an acoustic lens material.

### Example 3

At 25° C., 100 g RTV615 was added into a 250 mL flask. 10 g simethicone was used as a diluent, and stirred with 20 g polystyrene particles at 25° C. for 10 min, and a polystyrene particle dispersion was obtained after uniform mixing. A polystyrene particle of the polystyrene particles was spherical with a particle diameter of 1 μm. The polystyrene particle dispersion was added into the flask in four times, each time a quarter of the polystyrene particle dispersion was added, and stirred for 10 min. After the polystyrene particle dispersion was added, 10 g curing agent was added and stirred for 10 min, then poured into a mold, and placed in a 50% RH constant temperature and humidity box at 25° C. for curing 48 h to obtain modified silicone rubber. The modified silicone rubber may be used as an acoustic lens material.

### Example 4

At 25° C., 100 g RTV615 was added into a 250 mL flask. 20 g ethanol was used as a diluent, and stirred with 40 g polystyrene particles at 25° C. for 10 min, and a polystyrene particle dispersion was obtained after mixing uniformly. A polystyrene particle of the polystyrene particles was spherical with a particle diameter of 1 μm. The polystyrene particle dispersion was added into the flask in four times, each time a quarter of the polystyrene particle dispersion was added, and stirred for 10 min. After the polystyrene particle dispersion was added, dried in an oven at 50° C. for 2 h, an ethanol solvent was removed, 10 g curing agent was added and stirred for 10 min, then poured into a mold, and placed in a 50% RH constant temperature and humidity box at 25° C. for curing 48 h to obtain modified silicone rubber. The modified silicone rubber may be used as an acoustic lens material.

### Example 5

At 25° C., 100 g RTV615 was added into a 250 mL flask. 20 g ethanol was used as a diluent, and stirred with 40 g polystyrene particles at 25° C. for 10 min, and a polystyrene particle dispersion was obtained after mixing uniformly. A polystyrene particle of the polystyrene particles was spherical with a particle diameter of 10 μm. The polystyrene particle dispersion was added into the flask in four times, each time a quarter of the polystyrene particle dispersion was added, and stirred for 10 min. After the polystyrene particle dispersion was added, dried in an oven at 50° C. for 2 h, an ethanol solvent was removed, 10 g curing agent was added and stirred for 10 min, then poured into a mold, and placed in a 50% RH constant temperature and humidity box 25° C. for curing 48 h to obtain modified silicone rubber. The modified silicone rubber may be used as an acoustic lens material.

### Example 6

At 25° C., 100 g RTV615 was added into a 250 mL flask. 20 g simethicone was used as a diluent, stirred with 40 g polystyrene particles at 25° C. for 10 min, and mixed uniformly to obtain a polystyrene particle dispersion. A polystyrene particle of the polystyrene particles was spherical with a particle diameter of 1 μm. The polystyrene particle dispersion was added into the flask in four times, each time a quarter of the polystyrene particle dispersion was added, and stirred for 10 min. After the polystyrene particle dispersion was added, 10 g curing agent was added and stirred for 10 min, then poured into a mold, and placed in a 50% RH constant temperature and humidity box at 25° C. for curing 48 h to obtain modified silicone rubber. The modified silicone rubber may be used as an acoustic lens material.

### Example 7

At 25° C., 100 g RTV615 was added into a 250 mL flask. 30 g ethanol was used as a diluent, stirred with 60 g polystyrene particles at 25° C. for 10 min, and mixed uniformly to obtain a polystyrene particle dispersion. A polystyrene particle of the polystyrene particles was spherical with a particle diameter of 1 μm. The polystyrene particle dispersion was added into the flask in four times, each time a quarter of the polystyrene particle dispersion was added, and stirred for 10 min. After the polystyrene particle dispersion was added, dried in an oven at 50° C. for 2 h, an ethanol solvent was removed, 10 g curing agent was added and stirred for 10 min, then poured into a mold, and placed in a 50% RH constant temperature and humidity box at 25° C. for curing 48 h to obtain modified silicone rubber. The modified silicone rubber may be used as an acoustic lens material.

It can be understood that the modified silicone rubber or the acoustic lens material obtained in the examples may be further processed to produce an acoustic lens.

### Example 8

Example 8 was basically the same as Example 1, except that the polystyrene particles in Example 1 were replaced with polystyrene particles of the same mass and a particle diameter of 20 μm.

### Example 9

Example 9 was basically the same as Example 1, except that the polystyrene particles in Example 1 were replaced with polystyrene particles with the same particle diameter and a mass of 15 g.

### Contrast Example 1

At 25° C., 100 g RTV615 was added into a 250 mL flask. 10 g curing agent was added, stirred at 25° C. for 1 h, mixed uniformly, poured into a mold, and placed in a 50% RH constant temperature and humidity box at 25° C. for curing 48 h to obtain a cured matrix formed by curing the RTV silicone rubber which may be used as an acoustic lens material.

### Contrast Example 2

Contrast Example 2 was basically the same as Example 1, except that in Contrast Example 2, the polystyrene particles in Example 1 were replaced with 1 μm polystyrene particles and 10 μm polystyrene particles with the same total mass and a mass ratio of 1:1.

### Contrast Example 3

Contrast Example 3 was basically the same as Example 1, except that in Contrast Example 3, the polystyrene particles in Example 1 were replaced with 0.1 μm polystyrene particles with the same mass.

### Contrast Example 4

Contrast Example 4 was basically the same as Example 1, except that the polystyrene particles in Example 1 were replaced with 70 g polystyrene particles with the same particle diameter.

### Contrast Example 5

Contrast Example 5 was basically the same as Example 1, except that the polystyrene particles in Example 1 were replaced with 10 g polystyrene particles with the same particle diameter.

A part of parameters of each Example and Contrast Example and the surface Shore hardness, the acoustic impedance, the acoustic attenuation, and the acoustic reflection coefficient data of the modified silicone rubber and the cured matrix formed by curing the RTV silicone rubber may be shown in the following table:

| | RTV | | | | Surface | | Acoustic | Acoustic |
|---|---|---|---|---|---|---|---|---|
| | Silicone | Polystyrene | | Curing | Shore | Acoustic | attenuation | reflection |
| Group | rubber | particles | Diluent | agent | hardness | impedance | coefficient | coefficient |
| Example | 100 g | 20 g, | 10 g, | 10 g | 18.9 HA | 1.18MRayl | 16.8 | 1.43% |
| 1 | | 1 μm | Ethanol | | | | dB/cm | |
| Example | 100 g | 20 g, | 10 g, | 10 g | 19.6 HA | 1.16MRayl | 17.2 | 1.63% |
| 2 | | 10 μm | Ethanol | | | | dB/cm | |
| Example | 100 g | 20 g, | 10 g, | 10 g | 23.3 HA | 1.18MRayl | 17.3 | 1.43% |
| 3 | | 1 μm | Simethic | | | | dB/cm | |
| | | | one | | | | | |
| Example | 100 g | 40 g, | 20 g, | 10 g | 32.0 HA | 1.29MRayl | 19.1 | 0.57% |
| 4 | | 1 μm | Ethanol | | | | dB/cm | |
| Example | 100 g | 40 g, | 20 g, | 10 g | 32.5 HA | 1.27MRayl | 21.6 | 0.69% |
| 5 | | 10 μm | Ethanol | | | | dB/cm | |
| Example | 100 g | 40 g, | 20 g, | 10 g | 36.9 HA | 1.32MRayl | 19.9 | 0.41% |
| 6 | | 1 μm | Simethic | | | | dB/cm | |
| | | | one | | | | | |
| Example | 100 g | 60 g, | 30 g, | 10 g | 39.5 HA | 1.36MRayl | 26.9 | 0.24% |
| 7 | | 1 μm | Ethanol | | | | dB/cm | |
| Example | 100 g | 20 g, | 10 g, | 10 g | 19.7 HA | 1.16MRayl | 23.5 | 1.63% |
| 8 | | 20 μm | Ethanol | | | | dB/cm | |
| Example | 100 g | 15g, | 10 g, | 10 g | 17.4 HA | 1.13MRayl | 16.4 | 1.98% |
| 9 | | 1 μm | Ethanol | | | | dB/cm | |
| Contrast | 100 g | — | — | 10 g | 15.9 HA | 1.07MRayl | 15.4 | 5.52% |
| Example | | | | | | | dB/cm | |
| 1 | | | | | | | | |
| Contrast | 100 g | 20 g, | 10 g, | 10 g | 19.2 HA | 1.18MRayl | 29.4 | 1.43% |
| Example | | 1 μm + | Ethanol | | | | dB/cm | |
| 2 | | 10 μm | | | | | | |
| Contrast | 100 g | 20 g, | 10 g, | 10 g | 18.4 HA | 1.18MRayl | 31.0 | 1.43% |
| Example | | 0.1 μm | Ethanol | | | | dB/cm | |
| 3 | | | | | | | | |
| Contrast | 100 g | 70 g, | 10 g, | 10 g | 41.7 HA | 1.39MRayl | 34.7 | 0.14% |
| Example | | 1 μm | Ethanol | | | | dB/cm | |
| 4 | | | | | | | | |
| Contrast | 100 g | 10 g, | 10 g, | 10 g | 16.8 HA | 1.09MRayl | 16.3 | 2.51% |
| Example | | 1 μm | Ethanol | | | | dB/cm | |
| 5 | | | | | | | | |

A test standard or a test method of the surface Shore hardness is based on GB/T 531.1-2008. The test standard or test method of the acoustic impedance is based on YY/T 1668-2019. The test standard or test method for the acoustic attenuation at a frequency of 5 MHz is based on YY/T 1668-2019. The test standard or test method of the acoustic reflection coefficient is based on YY/T 1668-2019 to obtain the material acoustic impedance, and then calculate the acoustic reflection coefficient according to the sound intensity transmission coefficient formula based on the YY/T 1668-2019 standard.

Contrast Example 1 did not add the polystyrene particles for modification. The surface Shore hardness of the obtained cured matrix formed by curing the RTV silicone rubber was 15.9, the acoustic impedance was 1.07 MRayl, the acoustic attenuation at the frequency of 5 MHz was 15.4 dB/cm, and the acoustic reflection coefficient was 5.52%. It can be seen that the acoustic impedance of the cured matrix formed by curing the RTV silicone rubber without adding the polystyrene particles for modification in Contrast Example 1 was low, only 1.07 MRayl, and the acoustic reflection coefficient was as high as 5.52%.

Contrast Example 2 was a mixture of polystyrene particles with two different particle diameters, i.e., the particle diameters were not highly uniform. It can be known from the results in the table that the uneven particle diameters of the polystyrene particles had a great influence on the acoustic attenuation, and the acoustic attenuation at the frequency of 5 MHz reached 29.4 dB/cm, indicating that the acoustic intensity loss was relatively large, and seriously reducing the sensitivity of the ultrasonic probe.

The particle diameter of a polystyrene particle of the polystyrene particles in Contrast Example 3 was 0.1 μm, which was too small. Because the RTV silicone rubber has a certain viscosity and is difficult to penetrate between the polystyrene particles, and the polystyrene particles are difficult to disperse in the RTV silicone rubber, as a result, although the acoustic impedance of the prepared modified silicone rubber had improved, the acoustic attenuation had a greater increase, and the acoustic attenuation at the frequency of 5 MHz reached 31.0 dB/cm, thereby indicating that the acoustic intensity loss was large, and seriously reducing the sensitivity of the ultrasonic probe.

The mass of the polystyrene particles in Contrast Example 4 was 70 g, the modification effect on RTV silicone rubber was reduced, and the acoustic attenuation at the frequency of 5 MHz was significantly improved to 34.7 dB/cm, thereby indicating that the acoustic intensity loss was relatively large, and seriously reducing the sensitivity of the ultrasonic probe.

The mass of the polystyrene particles in Contrast Example 5 was 10 g, the modification effect on the RTV silicone rubber was reduced, and the acoustic reflection coefficient was significantly increased to 2.51%, thereby indicating that the acoustic transmittance was low, and affecting the quality of ultrasonic imaging.

The acoustic impedance of the modified silicone rubber prepared in Example 1-Example 9 was over 1.12 Mrayl, the acoustic attenuation at the frequency of 5 MHz was less than 28 dB/cm, the acoustic reflection coefficient was less than 2.1%, and the surface Shore hardness was within a range of 17-40.

By comparing Contrast Example 1 with Example 1, Example 2 and Example 8, it can be known that the acoustic attenuation coefficient of the modified silicone rubber prepared by adding the polystyrene particles to RTV615 was slightly improved, especially the increase of the acoustic attenuation coefficient of Examples 1-2 was small, while the surface Shore hardness and acoustic impedance increased significantly. It can be known from Example 1, Example 2, and Example 8 that when the particle diameter of a polystyrene particle of the polystyrene particles increased within a range of 1 μm-20 μm, for example, when the particle diameter increased to 20 μm, the diffraction effect of the ultrasonic waves in the corresponding modified silicone rubber was worse, and the acoustic attenuation coefficient slightly increased. Therefore, the particle diameter of a polystyrene particle of polystyrene particles may further preferably within a range of 1 μm-10 μm. In terms of hardness, the modification effect of the 1 μm polystyrene particles was lower than that of 10 μm and 20 μm polystyrene particles, but the acoustic impedance, the acoustic attenuation and acoustic reflection coefficient of the 1 μm polystyrene particles were better than those of the 10 μm polystyrene particles and the 20 μm polystyrene particles. Therefore, the 1 μm polystyrene particles can be preferably used.

By comparing Example 1 with Example 3, it can be known that the polystyrene particle dispersion prepared with the simethicone as the diluent may improve the hardness of the prepared modified silicone rubber to a certain extent, because the simethicone may participate in the curing reaction of RTV silicone rubber as an active monomer, thereby increasing the crosslinking density, and then increasing the hardness of the modified silicone rubber.

It can be known from Example 1, Example 4, Example 7 and Example 9 that increasing the mass of the polystyrene particles may further improve the acoustic impedance and the Shore hardness of the modified silicone rubber, while the acoustic attenuation can increase accordingly. Relatively speaking, the mass of the polystyrene particles in Example 7 was 60 g (or 60 parts by weight), and the increase rate of the acoustic attenuation of the modified silicone rubber was relatively large, indicating that if the mass of the polystyrene particles continues to increase, or when the mass of the polystyrene particles is too large, the acoustic performance of the modified silicone rubber may be seriously affected.

It can be seen from Example 6 that when simethicone was used as a diluent, 20 g (or 20 parts by weight) of simethicone was added, and 40 g (or 40 parts by weight) of polystyrene particles were added, the acoustic impedance value of the modified silicone rubber obtained in the present embodiment can be improved to 1.32 MRayl from 1.07 MRayl of the cured matrix formed by curing the RTV silicone rubber, which was closer to 1.5 MRayl of the human tissue; the acoustic reflection coefficient was reduced to 0.41%, indicating a higher acoustic permeability, meanwhile, the acoustic attenuation was increased from 15.4 dB/cm at the frequency of 5 MHz to 19.9 dB/cm, thereby indicating that the acoustic attenuation characteristics were little affected and still at a low attenuation level. The Shore hardness reached 36.9, thereby satisfying the service life and the skin affinity. Therefore, the modified silicone rubber in Example 6 was a highly practical acoustic lens material.

The technical effects of the embodiments of the present disclosure include, but are not limited to: (1) the polystyrene particles do not contain polar functional groups, do not agglomerate, and have good dispersibility. Under the action of high-speed stirring, the polystyrene particles may be uniformly dispersed in the RTV silicone rubber, which may avoid the problem of strong reflection of sound waves caused by the agglomeration of the modified silicone rubber, and may make the modified silicone rubber have low acoustic reflection coefficient; (2) the density of the polystyrene particles is 1.05 g/cm³, which is equivalent to that of the RTV silicone rubber. The polystyrene particles are configured as the modified fillers of the RTV silicone rubber. The polystyrene particles will not undergo phase separation of floating and sedimentation in the RTV silicone rubber. Thus, the polystyrene particles may have good compatibility with the RTV silicone rubber and may ensure the dispersion uniformity of the polystyrene particles in the cured matrix formed by curing the RTV silicone rubber after curing; (3) the acoustic impedance of the polystyrene particles is approximately 2.5 MRayl, and the acoustic attenuation is only 1.7 dB/cm at an acoustic frequency of 5 MHz, which has high acoustic impedance and low acoustic attenuation characteristics. The polystyrene particles with high-homogeneity particle diameter, i.e., the particle diameter CV of a polystyrene particle of the polystyrene particles is less than 3%, are adopted, and the composition ratio of the polystyrene particles with the cured matrix formed by curing the RTV silicone rubber is coordinated, thereby making the polystyrene particles fill in the cured matrix formed by curing the RTV silicone rubber and have excellent solid phase homogeneity, and have a low degree of scattering of ultrasonic waves; (4) compared with the cured matrix formed by curing the unmodified silicone rubber, the polystyrene particles may increase the acoustic impedance of the prepared modified silicone rubber, thereby improving its acoustic matching characteristics with the human tissue, reducing the acoustic reflection coefficient, improving the surface hardness of the modified silicone rubber, and also ensuring that the acoustic attenuation characteristics are kept at a low level; and (5) the modified silicone rubber prepared in the examples of the present disclosure may have both low acoustic attenuation and acoustic reflection coefficient, relatively good acoustic impedance matching characteristics and high hardness, and may be used as the acoustic permeable materials for the ultrasonic diagnostic device. The modified silicone rubber not only has a long service life, but also improves the imaging sensitivity and the imaging quality of the ultrasonic diagnostic device, and has relatively good practicability.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements and amendments to the present disclosure. These modifications, improvements, and amendments are intended to be suggested by the present disclosure, and are within the spirit and scope of the exemplary embodiments of the present disclosure.

Meanwhile, the present disclosure uses specific words to describe the embodiments of the present disclosure. For example, “one embodiment”, “an embodiment”, and/or “some embodiments” refer to a certain feature, structure or characteristic related to at least one embodiment of the present disclosure. Therefore, it should be emphasized and noted that references to “one embodiment” or “an embodiment” or “an alternative embodiment” two or more times in different places in the present disclosure do not necessarily refer to the same embodiment. In addition, certain features, structures or characteristics in one or more embodiments of the present disclosure may be properly combined.

In addition, unless clearly stated in the claims, the sequence of processing elements and sequences described in the present disclosure, the use of counts and letters, or the use of other names are not used to limit the sequence of processes and methods in the present disclosure. While the foregoing disclosure has discussed by way of various examples some embodiments of the invention that are presently believed to be useful, it should be understood that such detail is for illustrative purposes only and that the appended claims are not limited to the disclosed embodiments, but rather, the claims are intended to cover all modifications and equivalent combinations that fall within the spirit and scope of the embodiments of the present disclosure. For example, although the implementation of various components described above may be embodied in a hardware device, it may also be implemented as a software only solution, e.g., an installation on an existing server or mobile device.

In the same way, it should be noted that in order to simplify the expression disclosed in this disclosure and help the understanding of one or more embodiments of the invention, in the foregoing description of the embodiments of the present disclosure, sometimes multiple features are combined into one embodiment, drawings or descriptions thereof. This method of disclosure does not, however, imply that the subject matter of the disclosure requires more features than are recited in the claims. Rather, claimed subject matter may lie in less than all features of a single foregoing disclosed embodiment.

In some embodiments, counts describing the quantity of components and attributes are used. It should be understood that such counts used in the description of the embodiments use the modifiers “about”, “approximately” or “substantially” in some examples. Unless otherwise stated, “about”, “approximately” or “substantially” indicates that the stated figure allows for a variation of ±20%. Accordingly, in some embodiments, the numerical parameters used in the disclosure and claims are approximations that can vary depending upon the desired characteristics of individual embodiments. In some embodiments, numerical parameters should consider the specified significant digits and adopt the general digit retention method. Although the numerical ranges and parameters used in some embodiments of the present disclosure to confirm the breadth of the range are approximations, in specific embodiments, such numerical values are set as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A modified silicone rubber, comprising:
a cured matrix formed by curing a room temperature vulcanized silicone rubber; and
polystyrene particles dispersed in the cured matrix formed by curing the room temperature vulcanized silicone rubber.

2. The modified silicone rubber of claim 1, wherein the cured matrix formed by curing the room temperature vulcanized silicone rubber is within a range of 110-140 parts by weight; and the polystyrene particles is within a range of 15-60 parts by weight.

3. The modified silicone rubber of claim 2, wherein the cured matrix formed by curing the room temperature vulcanized silicone rubber is within a range of 110-140 parts by weight; and the polystyrene particles is within a range of 15-40 parts by weight.

4. (canceled)

5. The modified silicone rubber of claim 1, wherein a polystyrene particle of the polystyrene particles is spherical.

6. The modified silicone rubber of claim 1, wherein a particle diameter coefficient of variation (CV) of the polystyrene particles is less than 3%.

7. The modified silicone rubber of claim 1, a particle diameter of a polystyrene particle of the polystyrene particles is within a range of 1 μm-20 μm.

8. The modified silicone rubber of claim 7, wherein the particle diameter of the polystyrene particle of the polystyrene particles is within a range of 1 μm-10 μm.

9. The modified silicone rubber of claim 1, an acoustic attenuation coefficient of the modified silicone rubber at a frequency of 5 MHz is less than 28 dB/cm.

10. The modified silicone rubber of claim 1, wherein an acoustic impedance of the modified silicone rubber is greater than 1.12 Mrayl.

11. The modified silicone rubber of claim 1, wherein an acoustic reflection coefficient of the modified silicone rubber is less than 2.1%.

12. A method for preparing a modified silicone rubber, comprising:
preparing a polystyrene particle dispersion, wherein the polystyrene particle dispersion includes polystyrene particles and a diluent;
obtaining a mixture by mixing the polystyrene particle dispersion with a room temperature vulcanized silicone rubber; and
obtaining the modified silicone rubber by adding a curing agent into the mixture for curing, thereby dispersing the polystyrene particles in a cured matrix formed by curing the room temperature vulcanized silicone rubber.

13. The method of claim 12, wherein the diluent includes at least one of a reactive diluent and a non-reactive diluent.

14. The method of claim 12, wherein the diluent includes a non-reactive diluent, and the method further includes:
removing the non-reactive diluent from the mixture prior to adding the curing agent into the mixture for curing.

15. The method of claim 12, wherein based on 100 parts by weight of the room temperature vulcanized silicone rubber, the curing agent is 10 parts by weight, the polystyrene particles are within a range of 15-60 parts by weight, and the diluent is within a range of 10-30 parts by weight.

16. The method of claim 12, wherein the cured matrix formed by curing the room temperature vulcanized silicone rubber is within a range of 110-140 parts by weight, and the polystyrene particles are within a range of 15-60 parts by weight.

17-18. (canceled)

19. The method of claim 12, wherein a polystyrene particle of the polystyrene particles is spherical.

20. The method of claim 12, wherein a particle diameter coefficient of variation (CV) of the polystyrene particles is less than 3%.

21. The method of claim 12, a particle diameter of a polystyrene particle of the polystyrene particles is within a range of 1 μm-20 μm.

22. (canceled)

23. The method of claim 12, wherein an acoustic attenuation coefficient of the modified silicone rubber at a frequency of 5 MHz is less than 28 dB/cm.

24-27. (canceled)

28. An ultrasonic probe, comprising:
a probe body; and
an acoustic permeable component, wherein the acoustic permeable component is arranged on a surface of the probe body, the acoustic permeable component includes a modified silicone rubber, and the modified silicone rubber includes:
a cured matrix formed by curing a room temperature vulcanized silicone rubber; and
polystyrene particles dispersed in the cured matrix formed by curing the room temperature vulcanized silicone rubber.
